(19) United States
(12) Patent Application Publication
(10) Pub. No.: US 2022/0349822 A1
(43) Pub. Date: Nov. 3, 2022

(54) TEST DEVICE AND METHOD FOR TESTING THE RETROREFLECTION AND/OR FLUORESCENCE OF AN OBJECT

(71) Applicant: DIEMIETWAESCHE.DE GMBH + CO. KG, Villingen-Schwenningen (DE)

(72) Inventor: Michael WEUTHEN, Gosheim (DE)

(21) Appl. No.: 17/624,451

(22) PCT Filed: Jul. 1, 2020
(86) PCT No.: PCT/EP2020/068551

§ 371 Date: Jan. 3, 2022

(30) Foreign Application Priority Data
Jul. 2, 2019 (DE) 10 2019 117 858.4

(51) Int. Cl. G01N 21/64; G01N 21/55; G01N 21/27

(52) U.S. Cl. CPC G01N 21/64; G01N 21/55; G01N 21/274; G01N 2021/551; G01N 2201/0221; G01N 2201/062; G01N 2021/8845

(57) ABSTRACT

A hand-held test device and a method for testing an object having at least one retrore-flective region and at least one fluorescent region, e.g. an item of high-visibility clothing. The hand-held test device includes a white light LED, a UV LED, a connnon photoreceiver, and a control unit. In a reflection test mode, a reflective region is briefly irradiated with white light and reflects onto the photoreceiver. In at least one fluorescence test mode, ultraviolet light is emitted by the onto a fluorescent region, which generates fluorescent light of a corresponding colour via the fluorescence, which is received by the photoreceiver. In all test modes, a receive sig corresponding with the irradiation strength is generated at the photoreceiver and transmitted to the control unit for evaluation, e.g. for carrying out a threshold value comparison with a predefined threshold value for the receive signal. Every test mode can preferably have a separate predefined threshold value for this purpose.

### CROSS-REFERENCE TO RELATED APPLICATIONS

This application is a National Stage of PCT Application No. PCT/EP2020/068551 filed on Jul. 1, 2020, which claims priority to German Patent Application No. 10 2019 117 858.4 filed on Jul. 2, 2019, the contents each of which are incorporated herein by reference thereto.

### TECHNICAL FIELD

The invention refers to a hand-held test device that is configured to test a retro-reflection and/or a fluorescence of an object, as well as a method for carrying out such a test. The object is particularly a textile material, such as clothing, and particularly high-visibility clothing.

### BACKGROUND

Specific objects, particularly high-visibility clothing have to comply with requirements for retro-reflection of light, particularly white light and/or for fluorescence of the material. For high-visibility clothing these requirements are defined, e.g. in the standard ISO 20471. Besides high-visibility clothing, also other objects exist, the visibility of which is of importance, such as warning triangles, markings of construction sites, traffic signs, markings on vehicles, particularly emergency vehicles of security and rescue forces, etc.

EP 3 351 379 A1 describes the production of fluorescent textiles. After production the color characteristics are tested according to the Xenon-Test following section 5.2 of ISO 20471 and the luminance after Xenon-radiation is measured.

Portable color measurement apparatus is offered by company HunterLab under the registered trademark ,,MiniScan® EZ“. For carrying out the Xenon-Test this apparatus comprises a Xenon flash lamp. The color measurement is expected to be in conformity with CIE and to provide high precision spectral data.

From a presentation of the company 3M on Nov. 17, 2014 under the title “Innovation meets Industry” a method for luminance measurement is known in which a light source and a luminance measurement apparatus are arranged in a distance of about 20 m to a dummy. High-visibility clothing can be arranged on the dummy. The dummy can be rotated about 360° around the height axis, such that the high-visibility clothing can be measured along the circumference by means of this arrangement.

EP 3 327 627 describes a method and a device for visual quality control of textiles. For this at least one image of the textile object is captured and a part of the image is selected. The pixles in this part are evaluated with a predefined evaluation criteria and an evaluation value is determined therefrom. The evaluation value is compared with a threshold, wherein the comparison represents the result of the quality control.

The objects mentioned initially are subject to a certain wear. For example, the material of high-visibility clothing can be subject to wear due to washing. This may affect the visibility. Therefore, a need exists to check whether the indicated objects comply with the predefined requirements with regard to visibility. The previous devices and methods are cumbersome and/or require expensive devices. It can be considered as object of the present invention to allow testing of the retro-reflection and/or fluorescence of an object that can be carried out very quickly and easily also by personnel that is trained only little or untrained.

### BRIEF SUMMARY

A hand-held device that is configured to test a retro-reflection of an object in a reflection test mode and a fluorescence of the object in at least one fluorescence test mode, the hand-held test device including: a housing on which a user interface for selection of the reflection test mode or a fluorescence test mode is arranged; wherein the housing includes a window, the window being at least transparent for light in a predefined wavelength range on a front side; a test arrangement having a white light emitting diode, a UV-light emitting diode, a receiving circuit comprising a photoreceiver and a control unit is provided in the housing; wherein the white light emitting diode and the photoreceiver are arranged such that white light emitted by the white light emitting diode through the window impinges on the object and is reflected from the object and that the reflected light reflected from the object and entering through the window is received on the photoreceiver; wherein the UV-light emitting diode and the photoreceiver are arranged such that ultraviolet light emitted from the UV-light emitting diode through the window impinges on the object and that light emitted, due to fluorescence, from the object because of excitation with ultraviolet light is received on the photoreceiver; and wherein the control unit is configured to control the white light emitting diode in the reflection test mode and the UV-light emitting diode in the at least one fluorescence test mode for emission of light and to evaluate a receive signal of the receiving circuit that characterizes an irradiance of the light impinging on the photoreceiver.

A method for testing a retro-reflection and/or a fluorescence of an object by means of a hand-held test device including a housing that comprises a user interface and a window transparent for light at least in a predefined wavelength range on a front side, wherein a test arrangement having a white light emitting diode, a UV-light emitting diode, a receiving circuit including a photoreceiver and a control unit is arranged in the housing, the method including the following steps: selection of a reflection test mode or a fluorescence test mode by means of the user interface; control of the white light emitting diode in the reflection test mode, such that the white light emitting diode emits white light through the window onto the object and the photoreceiver receives reflected light emitted from the white light emitting diode, reflected on the object and entering through the window; control of the UV-light emitting diode in the fluorescence test mode, such that the UV-light emitting diode emits ultraviolet light through the window onto the object and the photoreceiver receives light emitted by the object due to fluorescence; and evaluating a receive signal of the receiving circuit that characterizes an irradiance of the light impinging on the photoreceiver.

The inventive hand-held test device is configured to be operated in at least two distinct test modes. In a reflection test mode the retro-reflection of an object can be tested. In at least one fluorescence test mode a fluorescence of an object can be tested. Particularly, multiple fluorescence test modes are provided for varicolored fluorescent objects, e.g. one fluorescence test mode for a yellow fluorescent object and one fluorescence test mode for an orange fluorescent object. In addition or as an alternative, also other or further fluorescence test modes for other or further colors can be provided, e.g. for a red fluorescent object and/or a green fluorescent object.

The hand-held test device is particularly configured to test objects comprising textile material, such as clothing. The hand-held test device can be particularly configured and/or calibrated to test high-visibility clothing according to ISO 20471.

The hand-held test device has a housing with a user interface for selection of the test mode. In one embodiment the user interface can comprise in addition an optical and/or acoustic output means for outputting the test result to a user. As optical output means at least one signal light means and/or a display or another arbitrary indication can be used.

The housing has a window on a front side. The window is at least light transparent in a predefined wavelength range. In the embodiment the window is at least light transparent for light wavelengths emitted by at least one light emitting diode of a test arrangement of the hand-held test device and for light wavelengths in the visible range. The housing is, apart from the window, substantially closed in a light-proof manner, such that light can incident into the interior of the housing substantially exclusively through the window.

The test arrangement is present in the housing having a and preferably exactly one light emitting diode and a and particularly exactly one UV-light emitting diode. The test arrangement further comprises a receiving circuit having a and particularly exactly one photoreceiver as well as a control unit. The control unit is configured for controlling the white light emitting diode and the UV-light emitting diode. The control unit is communicatively connected with the receiving circuit and configured to receive a receive signal of the receiving circuit.

The white light emitting diode and the photoreceiver are arranged in the housing, such that white light emitted by the white light emitting diode through the window impinges on the object to be tested, is reflected there, enters again through the window and is received on the photoreceiver. White light travels this light path, if an object to be tested is present in front of or on the front side of the housing and the reflection test mode has been selected and started.

The UV-light emitting diode and the photoreceiver are arranged such that ultraviolet light emitted by the UV-light emitting diode through the window impinges on the object and excites fluorescent material there. The light emitted by the object, due to the excitation, enters through the window and is received on the photoreceiver. Ultraviolet light travels this light path, if an object with fluorescent material is present in front of or on the front side of the housing and if the at least one fluorescence test mode has been selected and started.

Preferably the photoreceiver has an optical axis that passes through the window, particularly in the center. The optical axis of the photoreceiver can be orientated substantially orthogonal to the plane of the window.

The white light emitting diode can be arranged offset parallel to the optical axis of the photoreceiver in order to avoid direct radiation of white light on the photoreceiver. Particularly the white light emitting diode is located closer to the window than the photoreceiver.

According to the invention, the control unit is configured to control the white light emitting diode in the reflection test mode and the UV-light emitting diode in the at least one fluorescence test mode for emission of light. A receive signal of the receiving circuit characterizes the irradiance of light impinging on the photoreceiver. The receive signal is evaluated by the control unit, wherein the evaluation corresponds to the test result. The test result can be output or indicated to the user via the user interface. In the simplest case it can be output or indicated to the user whether the tested object complies with the requirements for retro-reflection of a retro-reflective material or with the requirements for luminance of a fluorescent material. For this the receive signal can be compared with the reference value, for example. The reference value can be, for example, a minimum value that has to be reached.

As an alternative or in addition to this, an output can be provided indicating a percentage or ratio of the defined reference value. For example, this indication can be a percent value.

Therefore, a precise determination of a measurement value, e.g. a luminance, is not necessary according to the invention. With the invention the retro-reflection as well as the fluorescence of an object, particularly of high-visibility clothing, can be tested in a very simple manner. The test duration is extremely short and is in the range of a few seconds. For example, if high-visibility clothing is washed in a laundry and is tested after washing, the test of all of the washed clothing can be carried out in a short period. In doing so, it can be guaranteed that high-visibility clothing that is used and washed multiple times, still provides sufficient security for the person carrying the high-visibility clothing.

The hand-held test device comprises a very simple configuration and can be realized cheaply. A white light emitting diode, a UV-light emitting diode and a photoreceiver are already sufficient as optical components of the test arrangement of the hand-held test device. The electrical and/or electronic circuit of the test arrangement can be configured with simple and cheap standard components.

The control unit is communicatively connected with the user interface. In a preferred embodiment the user interface comprises at least one input key and/or a display. For example, the user interface can have an input key for each selectable test mode.

Preferably, the control unit is configured to compare the receive signal with a reference value. The reference value can be predefined in the context of a calibration of the hand-held test device. The comparison result indicates particularly whether the object complies with a requirement in terms of retro-reflection and/or fluorescence or indicates the ratio between the actually tested object and the predefined reference value in the respective test mode.

The hand-held test device itself can be used for determination and/or definition of the reference value. For example, a calibration can be carried out by means of a calibration object or a calibration device, the characteristics of which are known. During testing of a known calibration object the receive signal can form the reference value that characterizes, for example, a minimum value for the retro-reflection (reflection test mode) or a minimum value for the fluorescence (fluorescence test mode). The subsequent tests of objects to be tested can then determine whether the fluorescence or the retro-reflection of a tested object reaches at least the respectively assigned reference value and/or the value of the ratio of the fluorescence or retro-reflection of a tested object relative to the respectively assigned reference value. For example, the ratio can be output as percent value via the user interface.

As an alternative to a calibration object, also a light emitting calibration device can be used, for example, for which the luminance and/or the wavelength of the emitted light is known or is adjustable. By means of such a calibration device, the receiving circuit can be calibrated without use of the light emitting diodes of the hand-held test device.

It is also possible to provide one corresponding calibration mode that can be activated by an authorized user for each test mode. Thereby the hand-held test device cannot only be calibrated once in the context of manufacturing, but also again later, e.g. in order to be able to compensate changes in the characteristic of the light emitting diodes and/or the photoreceiver or in order to adapt the hand-held test device to actual environmental conditions.

It is also possible to create or preset one or more further calibration values in addition to the reference value and to store them in the control unit. In doing so, for example, a characteristic diagram, a characteristic curve or another characteristic can be created. By storing of additional calibration values, also a non-linear behavior can be considered, for example. The accuracy can be increased.

As already explained, the comparison result can be output as test result via the user interface and particularly via the display. In a preferred embodiment the ratio of the receive signal in relation to the reference value of the actually used test mode is indicated.

It is advantageous, if the housing comprises an abutment surface on the front side that is configured to be brought into abutment during testing of the object. The abutment surface can surround the window completely. Thereby incidence of scattered light through the window is avoided and a defined distance of the photoreceiver and the light emitting diode to the object is set. The window or a cover plate of the window can be part of the abutment surface or can be arranged adjacent to the abutment surface and particularly offset backwards relative to the abutment surface.

Preferably the abutment surface can comprise opaque, elastic parts that surround the window completely. In doing so, unevennesses in the object to be tested can be compensated.

In a preferred embodiment the receiving circuit comprises a transmission amplifier. The photoreceiver can be connected to an input of the transmission amplifier. The receive signal is provided at an output of the transmission amplifier. The transmission amplifier can comprise an adjustable resistance in order to adjust the value range and particularly a voltage range for the receive signal. For example, the receive signal can vary in the range of 0 to 5 V depending on the irradiance of the light incident on the photoreceiver.

In a preferred embodiment the photoreceiver has a relative spectral sensitivity such that it creates no or only a negligibly small photocurrent, if exclusively ultraviolet light impinges on the photoreceiver. Thus, ultraviolet scattered light that is emitted by the UV-light emitting diode cannot or only negligibly affect the irradiance determined by the photoreceiver in the at least one fluorescence test mode. In a preferred embodiment the photoreceiver is not sensitive for incident light having a wavelength of less than 400 nm.

The control unit can be configured to activate the white light emitting diode and/or the UV-light emitting diode for the test for a predefined emission period. The emission period is particularly less than 1.0 seconds. Due to the short emission period, no thermal influences due to heat radiation onto the object to be tested are created.

The test of an object is carried out as follows:

The hand-held test device is arranged with a window in direction to the object. Subsequently, a test mode is selected via the user interface. Preferably the control unit starts the selected test mode automatically. The control unit activates the white light emitting diode in the reflection test mode and the UV-light emitting diode in the at least one fluorescence test mode. The receive signal created by the receiving circuit is evaluated in the control unit. Particularly it is compared with a predefined reference value. The comparison result can be output via the user interface, for example in an acoustic and/or optic manner.

### BRIEF DESCRIPTION OF THE DRAWINGS

Advantageous configurations of the invention are derived from the dependent claims, the description as well as the drawings. In the following, preferred embodiments of the invention are explained in detail based on the attached drawings. The drawings show:

FIG. 1 a schematic block-diagram-like illustration of an embodiment of a hand-held test device,

FIG. 2 a schematic illustration of an object to be tested that is particularly a part of high-visibility clothing,

FIG. 3 a schematic illustration of the use of the hand-held test device of FIG. 1 during testing of the object of FIG. 2 in a reflection test mode,

FIG. 4 a schematic illustration of the use of the hand-held test device of FIG. 1 during testing of the object of FIG. 2 in a fluorescence test mode,

FIG. 5 a circuit diagram of an embodiment of a test arrangement of the hand-held test device,

FIG. 6 a characteristic curve by means of which an evaluation of a receive signal created during the test can be carried out and

FIG. 7 a relative spectral sensitivity of an embodiment of a photoreceiver of the hand-held test device.

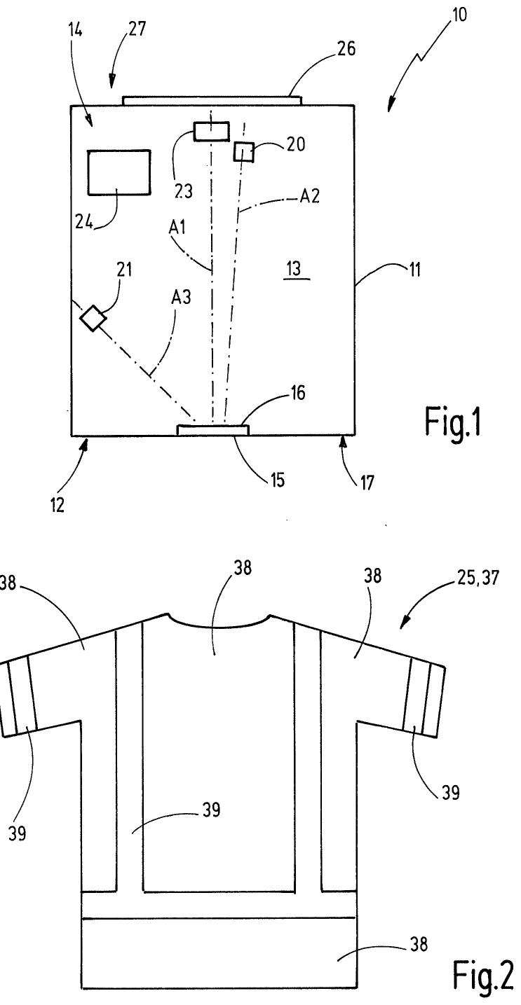

### DETAILED DESCRIPTION

In FIG. 4 an embodiment of a hand-held test device 10 is illustrated schematically in the manner of a block diagram. The hand-held test device 10 has a housing 11 having a front side 12. The housing 11 limits an interior 13 in which a test arrangement 14 of the hand-held test device 10 is arranged. The housing 11 has a window 15 on the front side 12 through which light at least in a predefined light wavelength range can incident into the interior 13. According to the example, the window 15 is formed by an opening in the housing 11 covered by a light permeable plate 16. In the embodiment the plate 16 is permeable at least for light in the ultraviolet wavelength range, as well as in the visible wavelength range. The plate 16 or window 15 can thus be permeable at least in a light wavelength range of 300 or 350 nm to 700 nm. The window 15 or plate 16 can also be transparent for all of the light wavelengths.

According to the example, light enters the interior 13 exclusively via window 15. Apart therefrom the interior 13 is closed against incident light by means of housing 11.

The housing 11 has an abutment surface 17 on the front side 12. The abutment surface 17 surrounds window 15 completely. The window 15 can be offset backward in direction toward a photoreceiver 23 relative to the abutment surface and/or can be arranged in the plane of the abutment surface 17 such that the surface of plate 16 facing outward is part of the abutment surface 17.

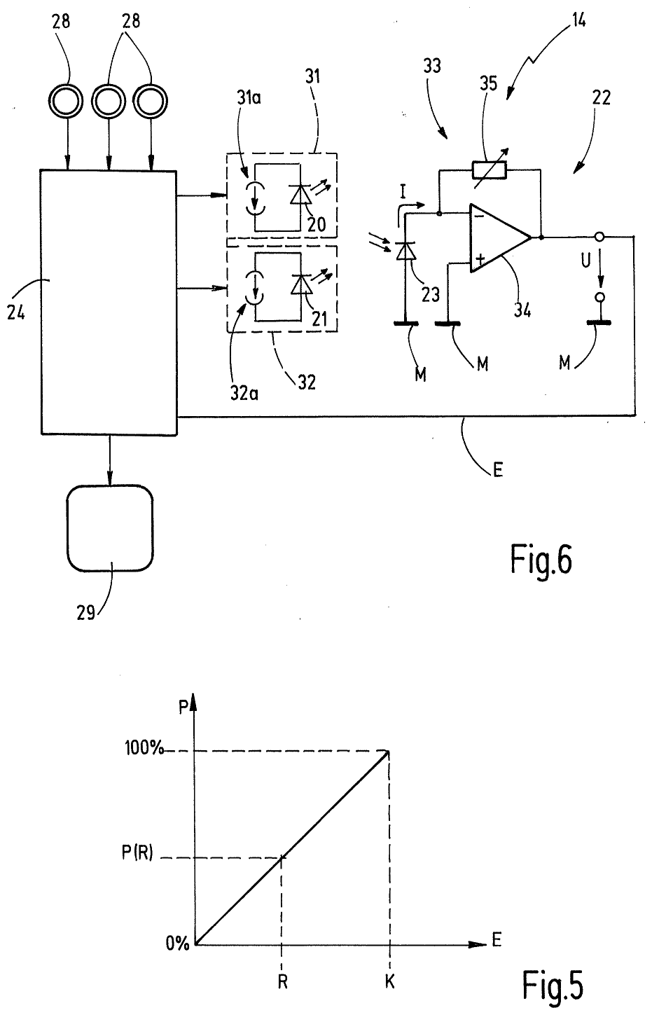

The test arrangement 14 of the hand-held test device 10 comprises a white light emitting diode 20, a UV-light emitting diode 21, a receiving circuit 22 (FIG. 5) having a photoreceiver 23 as well as a control unit 24. The white light emitting diode 20 is configured to emit white light, e.g. in a wavelength range of approximately 400 nm to approximately 700 nm. Light can be emitted by the white light emitting diode 20 that comprises preferably all of the wavelengths that can be captured by human eye.

The photoreceiver 23 has an optical axis A1 that passes through the window 15, e.g. approximately centrally. The optical axis A1 of photoreceiver 23 can be orientated orthogonal to the opening or window plane of window 15 and according to the example, orthogonal to plate 16.

The white light emitting diode 20 has an optical axis A2 that is arranged inclined with regard to the optical axis A1 of photoreceiver 23, e.g. in an angle of less than 10° and preferably in an angle of less than 8°. In the embodiment the optical axis A2 of the white light emitting diode 20 and the optical axis A1 of photoreceiver 23 include an angle of 7.5°. The optical axis A2 of the white light emitting diode 20 and the optical axis A1 of photoreceiver 23 intersect preferably in a point that is located in the plane of the abutment surface 17 on the front side 12 of housing 11 or is arranged close to this plane. At this location the surface of an object 25 to be tested is arranged during testing.

An optical axis A3 of the UV-light emitting diode 21 includes preferably an angle of 45° with the optical axis A1 of photoreceiver 23.

All of the optical axes A1, A2, A3 pass through window 15 or plate 16, as schematically illustrated in FIG. 1.

The hand-held test device 10 is configured to carry out distinct test modes. The operation is carried out by means of the control unit 24 in the selected test mode. The control unit 24 is communicatively coupled with a user interface 26. The user interface 26 is arranged on housing 11 accessible from outside, e.g. on a back side 27 of the housing opposite the front side 12. In the embodiment the user interface 26 comprises one selection key 28 for each possible test mode and has a means for outputting a test result, e.g. a display 29, that can be configured as screen (e.g. LED-, OLED- or LCD-screen). As an alternative or in addition, also other or further acoustical and/or optical output means can be present.

A circuit configuration for the test arrangement 14 is illustrated in FIG. 5 in the manner of a basic circuit diagram. The control unit 24 is communicatively connected with the user interface 26 and according to the example the selection keys 28, as well as the display 29. In addition, the control unit is communicatively connected with an operating circuit 31 for the white light emitting diode 20 and with an operating circuit 32 for the UV-light emitting diode 21. Each operating circuit 31, 32 comprises a controllable current source 31a or 32a and/or a controllable switch such that a current flow through the white light emitting diode 20 or the UV-light emitting diode 21 can be effected and/or the amount of which can be controlled due to the control by means of the control unit 24.

During first initial operation of hand-held test device 10 a current through the respective light emitting diode 20, 21 in the respective operating circuit 31, 32 has an amount of less than 100% or maximum 90% or maximum 80% of the maximum allowable operating current of the respective light emitting diode 20, 21. By increasing the current—for example by means of the respective controllable current source 31a, 32a—degradation effects of the light emitting diode 20, 21 that may occur with increasing total operation duration, can be compensated at least partly.

In the embodiment the receiving circuit 22 comprises a transmission amplifier 33 at the input of which the photoreceiver 23 is connected and at the output of which a receive signal E is provided that is applied in the form of an output voltage U with reference to a ground potential M according to the example. The transmission amplifier has an operational amplifier 34 according to the example to the inverting input of which the photoreceiver 23 is connected. In the embodiment the photoreceiver 23 is formed by a photodiode, the cathode of which is connected with the inverting input of the operational amplifier 34 and the anode of which is connected with ground potential M. Depending on the irradiance, the photoreceiver 23 provides a photo current I. A resistance and according to the example an adjustable resistance 35 is connected between the inverting input of the operational amplifier 34 and the output of the operational amplifier 34 at which the output voltage U applies. The adjustable resistance 35 is, for example, a potentiometer. By means of the adjustable resistance 35 the voltage range of the output voltage U can be adapted. For the output voltage U applies:

U=−R·I,

wherein R is the amount of the ohmic resistance of the adjustable resistance 35 and I is the photo current created by photoreceiver 23.

The receive signal E formed by the output voltage U, according to the example, is transmitted to the control unit 24. The receiving circuit 22 can comprise further circuit parts for processing of photo current I alternatively to the illustrated embodiment in order to create a receive signal E that can be received and evaluated by control unit 24. In the embodiment illustrated here the adjustable resistance 35 is adjusted such that the output voltage U varies in a range of 0 V to 5 V depending from the amount of the photo current I.

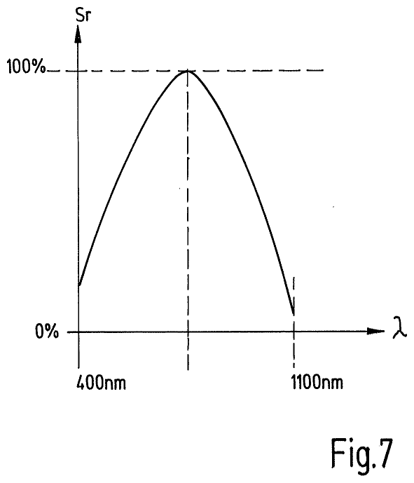

In FIG. 7 an exemplary relative spectral sensitivity Sr is illustrated for a photoreceiver 23. Preferably the relative spectral sensitivity Sr is selected, such that the photoreceiver 23 creates no or only a negligibley small photo current I, if ultraviolet light impinges on the photoreceiver 23. In the embodiment illustrated here the photoreceiver 23 delivers a photo current I that is larger than 0, if light impinges on the sensitive surface of the photoreceiver 23 in the wavelength range of minimum 400 nm to maximum approximately 1100 nm. In that ultraviolet light effects no or only a negligible photo current I, the test is not influenced by scattered light of the UV-light emitting diode 21. In order to avoid the influence of white scattered light of the white light emitting diode 20, it is offset in direction of the optical axis Al of photoreceiver 23 toward the window 15 such that a scattered light emission on the photoreceiver 23 is avoided or reduced such that it does not remarkably influence the photo current I.

In the example illustrated here the object 25 to be tested is a high-visibility clothing 37. With the hand-held test device 10 it can be tested whether the high-visibility clothing 37 fulfills defined requirements, particularly such requirements that are defined in the standard ISO 20471. In the embodiment the high-visibility clothing 37 has at least one fluorescent area 38 and at least one retro-reflective area 39. Preferably the entire high-visibility clothing 37 is made of a fluorescent textile carrier material and retro-reflective patches, e.g. strips, are attached onto the fluorescent textile carrier material.

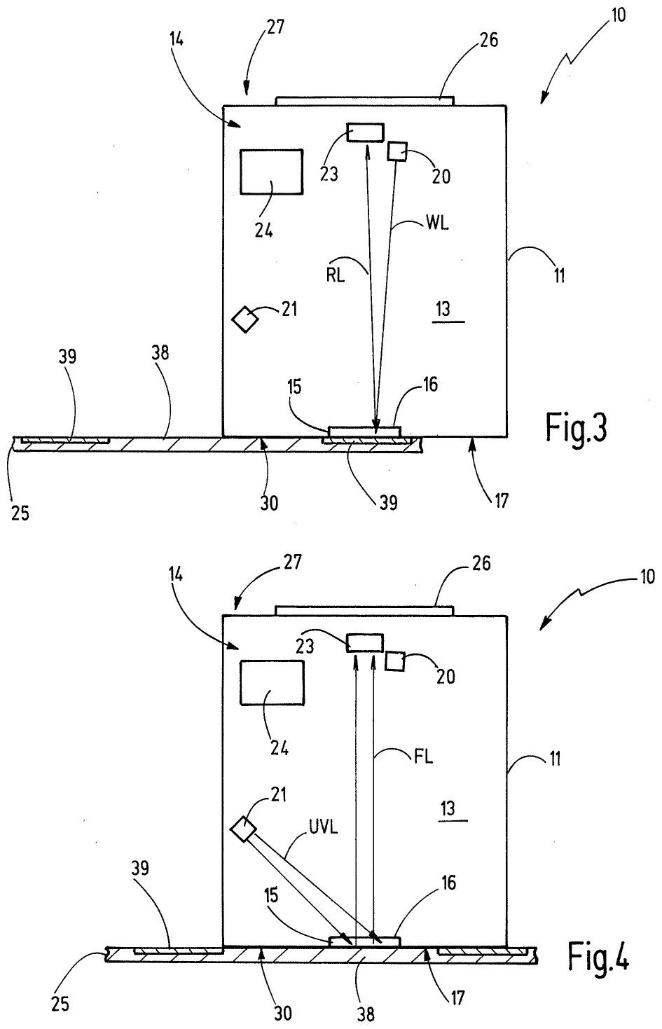

By means of the hand-held test device 10, the retro-reflection of the at least one retro-reflective area 39, as well as the fluorescence of the at least one fluorescent area 38 can be tested. For this the hand-held test device 10 comprises distinct test modes. In a reflection test mode the retro-reflection of a retro-reflective area 39 (FIG. 3) is tested and in the at least one fluorescence test mode, the fluorescence of a fluorescent area 38 (FIG. 4) is tested. In the embodiment the hand-held test device is configured to be able to test the fluorescence of a yellow fluorescent material, as well as of an orange fluorescent material and comprises for this purpose a separate fluorescence test mode in each case. Thus, in the embodiment three different test modes are present. In modification to this also more or less test modes can be present, e.g. yet an additional fluorescence test mode for red fluorescent material.

In FIG. 3 the execution of a test in a reflection test mode is schematically illustrated. For testing of a retro-reflective area 39, the hand-held test device 10 is placed with the abutment surface 17 onto the object 25 such that the window abuts against the retro-reflective area 39 to be tested or is located directly opposite thereto. In that the abutment surface 17 abuts against object 25, the entering of scattered light from the environment into the window 15 is avoided.

The control unit 24 activates the white light emitting diode 20 after selection of the reflection test mode via user interface 26 for a short emission period of, for example, less than 1 second. The white light emitting diode 20 emits white light WL during the emission period that exits through the window 15 and impinges onto the retro-reflective area 39 of object 25. The white light WL is reflected there and enters into window 15 as reflected light RL again and impinges onto the photoreceiver 23. Depending on the irradiance, the photoreceiver 23 creates a photo current I in the receiving circuit 22 that creates an output voltage U therefrom that is proportional to the amount of the photo current I in the embodiment. The output voltage U is provided to the control unit 24 as receive signal E. The UV-light emitting diode 21 is inactive in the reflection test mode.

Analog to this procedure, the abutment surface 17 is placed onto the object 25 for measuring a fluorescent area 38, such that a fluorescent area 38 abuts against window 15 or is directly located opposite window 15 (FIG. 4).

After selection of the current fluorescence test mode by means of the user interface 26 depending on the color of the fluorescent area 38 to be tested, the control unit 24 activates the UV-light emitting diode 21 for the emission period of less than 1 second according to the example. The UV-light emitting diode 21 emits ultraviolet light UVL during the emission period through window 15 onto the fluorescent area 38 to be tested. The fluorescent material is excited there, due to the impinging ultraviolet light UVL and emits fluorescent light FL that enters into housing 11 through window 15 and impinges onto photoreceiver 23. In doing so, a photo current I and a receive signal E proportional to the amount of the photo current I is created and transmitted to the control unit 24.

In the embodiment the test or evaluation in each test mode is carried out by comparison of the receive signal E with at least one reference value R stored in the control unit 24. This reference value R corresponds, for example, to a receive signal E that is created during testing of a reference object. The reference object comprises a known characteristic with regard to retro-reflection or fluorescence. For each test mode a separate reference value R is predefined that is used during testing for comparison.

Based on a predefined correlation, e.g. a linear correlation, a receive signal E created during testing of an object 25 to be tested can be compared with the stored reference value R of the respective test mode and from the comparison a test result can be created and output. The test result indicates whether and/or to which degree the tested characteristic (retro-reflection or fluorescence) of the object to be tested corresponds to the corresponding characteristic of the reference object characterized by the reference value of the test mode.

In the embodiment a percent value P is output on display 29 that is characteristic for the ratio of the actual receive signal relative to the reference value of the respective test mode. For example, the reference value R can alternatively describe a retro-reflection value of a retro-reflective material or a luminance of a fluorescent material of a reference object that only just complies with the requirements, to which a percent indication is assigned, e.g. 50%. If an object 25 to be tested creates a receive signal E that at least corresponds to the reference value R, it can be apparent from the output percent indication P (≥50%) that the object to be tested fulfills the defined requirements. If the receive signal E is less than the reference value R, it is apparent from the percent indication P (<50%) that the object to be tested does not comply with the requirements.

Therefore, the hand-held test device 10 can carry out a threshold comparison so-to-speak and indicate whether and/or to which degree the requirements are fulfilled or not. In the simplest case only the compliance with the requirements or non-compliance with the requirements indicating two-stage output can be carried out by means of the user interface 26. The output of a percent indication P has the advantage that also a tendency can be determined whether the object 25 complies only just with the requirements or is qualitatively still very well. As illustrated in FIG. 6, in the embodiment a linear characteristic curve is assumed the progress of which can be determined from known correlations or can be determined empirically. Also other characteristic curves can be used for converting the receive signal E in an indicated value, e.g. the percent indication P.

Moreover, during calibration of the hand-held test device 10 one or more additional calibration values K can be determined in addition to the reference value R. For example, a calibration value K can be determined on a reference object that has optimum characteristics in terms of the retro-reflection or the fluorescence. By recording additional calibration values K, the accuracy of the characteristic line can be improved.

At least one reference value R is stored for each test mode for comparison with the receive signal E in the respective test mode. This is advantageous, because the photoreceiver 23 has a wavelength-dependent characteristic that creates different photo currents I in case of different spectra in the distinct test modes. In doing so, the accuracy of the evaluation in the different test modes can be improved. A correlation schematically illustrated in FIG. 6 is thus stored for each test mode in the control unit 24.

The invention refers to a hand-held test device 10 and a method for testing an object 25 having at least one retro-reflective area 39 and at least one fluorescent area 38, e.g. a high-visibility clothing 37. The hand-held test device 10 comprises a white light emitting diode 20, a UV-light emitting diode 21, a common photoreceiver 23, as well as a control unit 24. In a reflection test mode a reflective area 39 is irradiated with white light WL for a short period and reflected onto the photoreceiver 23. In the at least one fluorescence test mode ultraviolet light UVL is emitted by means of the UV-light emitting diode onto the fluorescent area 38 that creates fluorescent light FL of a corresponding color, due to the fluorescence that is received by photoreceiver 23. In all test modes a receive signal E corresponding to the irradiance is created in the photoreceiver 23 and transmitted to the control unit 24 for evaluation, e.g. for carrying out a threshold comparison with a predefined threshold for the receive signal E. Each test mode can preferably comprise a separate predefined threshold for this purpose.

### LIST OF REFERENCE SIGNS

10 hand-held test device

11 housing

12 front side of housing

13 interior

14 test arrangement

15 window

16 plate

17 abutment surface

20 white light emitting diode

21 UV-light emitting diode

22 receiving circuit

23 photoreceiver

24 control unit

25 object

26 user interface

27 back side of housing

28 selection key

29 display

31 operating circuit for white light emitting diode

31a current source of operating circuit for white light emitting diode

32 operating circuit for UV-light emitting diode

32a current source of operating circuit for UV-light emitting diode

33 transmission amplifier

35 adjustable resistance

37 high-visibility clothing

38 fluorescent area

39 retro-reflecting area

A1 optical axis of photoreceiver

A2 optical axis of white light emitting diode

A3 optical axis of UV-light emitting diode

E receive signal

FL light emitted by fluorescence

K calibration value

M ground potential

P percent value

R reference value

RL reflected light

Sr relative spectral sensitivity

U output voltage

UVL ultraviolet light

WL white light

1. A hand-held test device that is configured to test a retro-reflection of an object in a reflection test mode and a fluorescence of the object in at least one fluorescence test mode, the hand-held test device comprising:
a housing on which a user interface for selection of the reflection test mode or a fluorescence test mode is arranged;
wherein the housing comprises a window, the window being at least transparent for light in a predefined wavelength range on a front side;
a test arrangement having a white light emitting diode, a UV-light emitting diode, a receiving circuit comprising a photoreceiver and a control unit is provided in the housing;
wherein the white light emitting diode and the photoreceiver are arranged such that white light emitted by the white light emitting diode through the window impinges on the object and is reflected from the object and that the reflected light reflected from the object and entering through the window is received on the photoreceiver;
wherein the UV-light emitting diode and the photoreceiver are arranged such that ultraviolet light emitted from the UV-light emitting diode through the window impinges on the object and that light emitted, due to fluorescence, from the object because of excitation with ultraviolet light is received on the photoreceiver; and
wherein the control unit is configured to control the white light emitting diode in the reflection test mode and the UV-light emitting diode in the at least one fluorescence test mode for emission of light and to evaluate a receive signal of the receiving circuit that characterizes an irradiance of the light impinging on the photoreceiver.

2. The hand-held test device according to claim 1, wherein the hand-held test device is configured to test in a first fluorescence test mode a fluorescence of a yellow fluorescent material of the object and in a second fluorescence test mode a fluorescence of an orange fluorescent material of the object.

3. The hand-held test device according to claim 1, wherein the user interface comprises a display that is communicatively coupled with the control unit.

4. The hand-held test device according to claim 1, wherein the control unit is configured to compare the receive signal with a reference value.

5. The hand-held test device according to claim 4, wherein a comparison of the receive signal and the reference value indicates whether the object reflects sufficient light or is sufficiently fluorescent.

6. The hand-held test device according to claim 4, wherein the reference value is created by testing of at least one calibration device or a calibration object with the hand-held test device and the reference value is stored in the control unit.

7. The hand-held test device according to claim 6, wherein in addition to the reference value, at least one additional calibration value is created by testing the calibration device or at least one additional calibration object by means of the hand-held test device and the at least one additional calibration value is stored in the control unit.

8. The hand-held test device according to claim 5, wherein the user interface comprises a display that is communicatively coupled with the control unit and the comparison of the receive signal and the reference value is outputted by the display.

9. The hand-held test device according to claim 1, wherein the housing comprises an abutment surface on the front side that is configured to be brought into abutment with the object during testing.

10. The hand-held test device according to claim 9, wherein the light transparent window is arranged adjacent to the abutment surface or is at least a part of the abutment surface.

11. The hand-held test device according to claim 1, wherein the receiving circuit comprises a transmission amplifier, and the photoreceiver is connected to an input of the transmission amplifier and the receive signal is provided at an output of the transmission amplifier.

12. The hand-held test device according to claim 11, wherein the transmission amplifier comprises an adjustable resistance.

13. The hand-held test device according to claim 1, wherein the photoreceiver comprises a relative spectral sensitivity, such that the photoreceiver creates no or only a negligible photo current, if ultraviolet light emitted from the UV-light emitting diode impinges on the photoreceiver.

14. The hand-held test device according to claim 1, wherein the control unit is configured to activate the white light emitting diode and/or the UV-light emitting diode for a predefined emission period.

15. A method for testing a retro-reflection and/or a fluorescence of an object by means of a hand-held test device comprising a housing that comprises a user interface and a window transparent for light at least in a predefined wavelength range on a front side, wherein a test arrangement having a white light emitting diode, a UV-light emitting diode, a receiving circuit comprising a photoreceiver and a control unit is arranged in the housing, the method comprising the following steps:
selection of a reflection test mode or a fluorescence test mode by means of the user interface;
control of the white light emitting diode in the reflection test mode, such that the white light emitting diode emits white light through the window onto the object and the photoreceiver receives reflected light emitted from the white light emitting diode, reflected on the object and entering through the window;
control of the UV-light emitting diode in the fluorescence test mode, such that the UV-light emitting diode emits ultraviolet light through the window onto the object and the photoreceiver receives light emitted by the object due to fluorescence; and
evaluating a receive signal of the receiving circuit that characterizes an irradiance of the light impinging on the photoreceiver.

16. The hand-held test device according to claim 2, wherein the user interface comprises a display that is communicatively coupled with the control unit.

17. The hand-held test device according to claim 16, wherein the control unit is configured to compare the receive signal with a reference value.

18. The hand-held test device according to claim 17, wherein a comparison of the receive signal and the reference value indicates whether the object reflects sufficient light or is sufficiently fluorescent.

19. The hand-held test device according to claim 18, wherein the reference value is created by testing of at least one calibration device or a calibration object with the hand-held test device and the reference value is stored in the control unit.

20. The hand-held test device according to claim 19, wherein in addition to the reference value, at least one additional calibration value is created by testing the calibration device or at least one additional calibration object by means of the hand-held test device and the at least one additional calibration value is stored in the control unit.
